(19) United States
(12) Patent Application Publication
(10) Pub. No.: US 2016/0051569 A1
(43) Pub. Date: Feb. 25, 2016

(54) Methods for Inhibiting the Development, Formation and/or Maturation of Bacterial and Fungal Biofilms

(71) Applicant: Professional Compounding Centers of America (PCCA), Houston, TX (US)

(72) Inventor: Daniel Banov, Sugar Land, TX (US)

(21) Appl. No.: 14/831,695

(22) Filed: Aug. 20, 2015

Related U.S. Application Data
(60) Provisional application No. 62/039,818, filed on Aug. 20, 2014.
(60) Provisional application No. 62/039,768, filed on Aug. 20, 2014.
(60) Provisional application No. 62/039,774, filed on Aug. 20, 2014.
(60) Provisional application No. 62/039,799, filed on Aug. 20, 2014.

(51) Int. Cl. A61K 31/616; A61K 31/44; A61K 9/00; A61K 31/416; A61K 31/407; A61K 31/4015; A61K 31/5415; A61K 31/426

(52) U.S. Cl. CPC A61K 31/616; A61K 31/5415; A61K 31/44; A61K 31/426; A61K 31/416; A61K 31/407; A61K 31/4015; A61K 9/0014

(57) ABSTRACT

The present disclosure describes compositions and methods for inhibiting the development, formation and/or maturation of bacterial and fungal biofilms. The biofilm inhibiting compositions include non-steroidal anti-inflammatory drugs (NSAIDs) as APIs, and ethoxylated oil as a solubilizing agent. The ethoxylated oils employed within the biofilm inhibiting compositions includes between 9 and 24 ethoxylations/molecules. The NSAIDs agents within the biofilm inhibiting compositions inhibit the syntheses of prostaglandins by blocking the cyclooxygenase (COX) enzyme system due to the analgesic, anti-inflammatory, and antipyretic properties of the NSAIDs. The biofilm inhibiting compositions inhibit the cyclooxygenase isoenzymes required for prostaglandin formation, thereby preventing the colonization of fungi or bacteria as well as the formation of biofilms.

### CROSS-REFERENCE TO RELATED APPLICATIONS

This application claims priority to U.S. Provisional Application Ser. No. 62/039,818, filed Aug. 20, 2014, U.S. Provisional Application Ser. No. 62/039,768, filed Aug. 20, 2014, U.S. Provisional Application Ser. No. 62/039,774, filed Aug. 20, 2014, and U.S. Provisional Application Ser. No. 62/039,799, filed Aug. 20, 2014, which are hereby incorporated by reference.

### BACKGROUND

1. Field of the Disclosure

The present disclosure relates generally to microbial infections, and more particularly, to methods for inhibiting the development, formation and/or maturation of bacterial and fungal biofilms using pharmaceutical compositions.

2. Background Information

Biofilms are known as populations of bacteria or fungi growing attached to an inert or living surface. There has been evidence that biofilms may constitute a significant threat to human health. The US Public Health Service estimates that biofilms may be responsible for more than 80% of bacterial infections in humans. Further, biofilms may cause a great variety of diseases, such as, for example dental caries, periodontitis, cystic fibrosis pneumonia, native valve endocarditis, and otitis media as well as infection of various medical devices, such as, for example urinary catheters, mechanical heart valves, cardiac pacemakers, prosthetic joints, and contact lenses, amongst others.

Candida albicans is a fungal pathogen that causes biofilms of clinical significance, which can in turn cause a plurality of diseases ranging from simple benign infections to life threatening conditions. An interesting aspect of Candida albicans pathogenesis is the ability of both the host and pathogen to produce prostaglandins that are potent immunomodulators, which can contribute to biofilm formation. Prostaglandins are known to be produced by Candida albicans and play an important role in fungal colonization and thus in the formation of biofilms.

Several strategies have been developed to overcome said diseases and infections. For instance, the use of coatings for medical devices that are either antibacterial or nonbiofuling. However, these strategies have shown low efficacy in preventing the formation of biofilms. Also, other approaches to eliminate biofilms have focused on dispersion compounds to entice the bacteria or fungus to actively escape or disperse from the biofilm, where the bacteria become more susceptible to antibiotics. Additionally, another method includes isolated proteins and active fragments and variants thereof that promote detachment of bacterial or fungal cells from a biofilm. Further methods include mutating the bacterial cells to inhibit detachment of bacterial cells from biofilms.

Bacteria or fungi growing in biofilms exhibit increased resistance to antimicrobial agents and are very difficult to eradicate. Due to the difficulty in eradicating formed biofilms, a method for inhibiting the development, formation and/or maturation of bacterial or fungal biofilms is desirable.

### SUMMARY

The present disclosure describes compositions and methods for inhibiting the development, formation and/or maturation of bacterial or fungal biofilms. In some embodiments, the biofilm inhibiting compositions include active pharmaceutical ingredients (APIs), such as non-steroidal anti-inflammatory drugs (NSAIDs), and a solubilizing agent.

In some embodiments, suitable NSAIDs include acetylsalicylic acid, bucloxic acid, flufenamic acid, mefanamic acid, niflumic acid, tiaprofenic acid, tolfenamic acid, bendazac, carprofen, ketoprofen, diclofenac, diflunisal, etodolac, fenbufen, fenoprofen, fentiazac, flurbiprofen, isoxicam, naproxen, pirfrofen, piroxicam, sulindac, suprofen, tenoxicam, tolmetin, zomepirac, among other suitable NSAIDs.

In some embodiments, the analgesic, anti-inflammatory, and antipyretic action of NSAIDs is believed to be the result of their ability to block the cycle-oxygenase (COX) enzyme system, thereby inhibiting the syntheses of prostaglandins from the precursor, arachidonic acid. An interesting aspect of Candida albicans pathogenesis is the ability of both the host and pathogen to produce prostaglandins, which are potent immunomodulators. Prostaglandins can enhance biofilm development by increasing adhesion, germination, biofilm mass, matrix, and shed cell population, thereby playing an important role in fungal colonization.

In some embodiments, suitable solubilizing agents include ethoxylated oils. In these embodiments, the ethoxylated oils within the biofilm inhibiting compositions are PEGylated oils. Further to these embodiments, the PEGylated oils are surfactants that possess increased solubilizing properties. In these embodiments, the PEGylated oils include between 9 and 24 ethoxylations/molecules. Further to these embodiments, the PEGylated oils are employed to improve the APIs solubility, stability, and the delivery efficacy of a particular NSAID agent to inhibit the development, formation and/or maturation of bacterial and fungal biofilms.

In an example, the biofilm inhibiting compositions include from about 0.1% w/w to 5% w/w of and NSAID agent, preferably from about 2% w/w to 5% w/w; and from about 1% w/w to 20% w/w of ethoxylated oils, preferably from about 5% w/w to 10% w/w.

In some embodiments, the biofilm inhibiting compositions are administered on the body surface of a patient in various dosage forms, such as, for example ointments, creams, gels, lotions, solutions, and pastes. In these embodiments, suitable vehicles are employed for each dosage form. Further to these embodiments, the biofilm inhibiting compositions are applied on the body surface of a patient within a dosage ranging from about 500 mg/day to 5000 mg/day, preferably from about 1000 mg/day to 3000 mg/day.

Numerous other aspects, features of the present disclosure may be made apparent from the following detailed description.

### DETAILED DESCRIPTION

The present disclosure is here described in detail. Other embodiments may be used and/or other changes may be made without departing from the spirit or scope of the present disclosure. The illustrative embodiments described in the detailed description are not meant to be limiting of the subject matter presented here.

### Definitions

As used here, the following terms have the following definitions:

“Active Pharmaceutical Ingredients (APIs)” refer to chemical compounds that induce a desired effect, and include agents that are therapeutically effective, prophylactically effective, or cosmeceutically effective.

“Biofilm” refers to a structured consortium of bacteria of fungi embedded in a self-produced polymer matrix consisting of polysaccharides, protein, and DNA.

“Body surface” refers to the skin of a mammal.

Inhibit” refers to decrease, limit, or block the action or function of a process.

“Non-steroidal anti-inflammatory drugs (NSAIDs)”, or equivalently, “non-steroidal anti-inflammatory agents/analgesics” refer to drugs with the ability to block the cycle-oxygenase (COX) enzyme system.

“Patient” refers to warm-blooded animals, such as mammals, for example, humans, who are in need of treatment.

“Surfactants” refer to compounds that reduce surface tension of liquids, and are shorter names for surface active agents.

“Therapeutically effective amount” refers to the amount of subject compound that will elicit the biological or medical response of a tissue, system, animal or human that is being sought.

“Treating” and “Treatment” refers to reduction in severity and/or frequency of symptoms, elimination of symptoms and/or underlying cause, prevention of the occurrence of symptoms and/or their underlying cause, and improvement or remediation of damage.

### DESCRIPTION OF THE DISCLOSURE

The present disclosure is directed towards biofilm inhibiting compositions that include non-steroidal anti-inflammatory drug (NSAID) and ethoxylated oils. Further, the present disclosure provides a method for inhibiting the development, formation and/or maturation of bacterial or fungal biofilms, comprising topically administering to the body surface of the patient in need thereof a therapeutically effective amount of the biofilm inhibiting composition as described below and elsewhere herein.

### Formulation

In some embodiments, the biofilm inhibiting compositions include at least one non-steroidal anti-inflammatory drug (NSAID) as active pharmaceutical ingredient (API), and a solubilizing agent, such as ethoxylated oils, among other suitable ingredients.

In an example, the biofilm inhibiting compositions include from about 0.1% w/w to 5% w/w of the NSAID agent, preferably from about 2% w/w to 5% w/w; and from about 1% w/w to 20% w/w of ethoxylated oils, preferably from about 5% w/w to 10% w/w.

Non-Steroidal Anti-Inflammatory Drugs (NSAIDs)

NSAIDs are widely used for the treatment of a variety of conditions associated with pain, fever and inflammation, including osteoarthritis, rheumatoid arthritis, gout, and ankylosing spondylitis. NSAIDs are also widely used for treating acute pain associated with injuries and surgical procedures (including dental procedures) and headaches. The beneficial effects of NSAIDs are largely believed to be attributable to their ability to suppress prostaglandin synthesis by inhibiting cyclooxygenase-1 (COX-1) and cyclooxygenase-2 (COX-2).

The analgesic, anti-inflammatory, and antipyretic properties of NSAIDs are believed to be the result of the ability of NSAIDs agents to block the cyclooxygenase (COX) enzyme system, thereby inhibiting the syntheses of prostaglandins from the arachidonic acid precursor. Inhibition of the cyclooxygenase isoenzymes required for prostaglandin formation may prevent the colonization of fungi or bacteria, and thus, the formation of biofilms.

In some embodiments, the NSAIDs employed within the biofilm inhibiting compositions include acetylsalicylic acid, bucloxic acid, flufenamic acid, mefanamic acid, niflumic acid, tiaprofenic acid, tolfenamic acid, bendazac, carprofen, ketoprofen, diclofenac, diflunisal, etodolac, fenbufen, fenoprofen, fentiazac, flurbiprofen, isoxicam, naproxen, pirfrofen, piroxicam, sulindac, suprofen, tenoxicam, tolmetin, and zomepira, among others.

Ethoxylated Oil

Ethoxylation

Ethoxylation is a chemical process used to create surfactants. The process of ethoxylation involves adding ethylene oxide to an alcohol or phenol compound to produce surfactants, such as ethoxylated oils. In some embodiments, the ethoxylated oils employed within the biofilm inhibiting compositions are PEGylated oils. In these embodiments, the PEGylated oils include between 9 and 24 ethoxylations/molecules. Further to these embodiments, the PEGylated oils provide improved drug solubility and increased drug stability.

In some embodiments, the improved drug solubility increases the penetration of the NSAIDs, thereby enhancing the delivery efficacy of said NSAIDs. In these embodiments, the enhanced penetration of the NSAIDs enables a desired concentration of the APIs in the site of action, thereby improving the pharmacological response. Further to these embodiments, the PEGylated oils employed within the biofilm inhibiting compositions exhibit bacterial efflux pumps inhibitor properties and also reduce the water activity of the bacterial cells, thereby enhancing the therapeutic effectiveness.

In other embodiments, the biofilm inhibiting compositions are employed for inhibiting the development, formation and/or maturation of bacterial and fungal biofilms, such as Candida albicans biofilms, among others.

Candida albicans and Prostaglandins

Prostaglandins are lipid compounds that are derived from a fatty acid known as arachidonic acid (AA). In the mammalian cell, cyclooxygenase (COX) isoenzymes, COX-1 and COX-2 facilitate the conversion of AA to prostaglandins including prostaglandin E2 (PGE2). Depending on the target cell and receptor, PGE2 can act as an anti-inflammatory or pro-inflammatory mediator of the immune system.

Candida albicans also produce a compound that cross-reacts with PGE2 called PGEx (prostaglandin cross reactive compound) that acts on host-cells functionally similar to PGE2. PGE2 and PGEx enhance biofilm development by increasing adhesion, germination, biofilm mass, matrix, and shed cell population. Further, Candida albicans biofilms produce more prostaglandins than planktonic cells.

Prostaglandin inhibitors inhibit COX enzymes in mammalian cells. Treatment of Candida with COX inhibitors inhibits fungal PGE2 production. Moreover, COX inhibitors have been shown to decrease Candida albicans biofilm formation in vitro.

In other embodiments, various additives are included within the biofilm inhibiting compositions to facilitate the preparation of suitable forms. For example, additives include humectants, pH adjusting agents, preservatives, antioxidants, silk amino acids, enzymes, treintoin, vitamins, fragrance, colorant, gelling agents, and stabilizers, among others.

### Administration

In some embodiments, the biofilm inhibiting compositions are administered on the body surface of a patient in various dosage forms, such as, for example ointments, creams, gels, lotions, solutions, and pastes. In these embodiments, suitable vehicles are employed for each dosage form. Further to these embodiments, the biofilm inhibiting compositions are applied on the body surface of a patient within a dosage ranging from about 500 mg/day to 5000 mg/day, preferably from about 1000 mg/day to 3000 mg/day.

In some embodiments, the biofilm inhibiting compositions are employed before and after surgeries to inhibit or prevent bacterial or fungal infections. In an example, the biofilm inhibiting compositions are employed before and after transplantations procedures, immunosuppression, the use of chronic indwelling devices (e.g., dental implants, catheters, heart valves, vascular bypass grafts, ocular lenses, artificial joints, and central nervous system shunts), and prolonged intensive care unit stays.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A method of inhibiting a biofilm comprising applying a topical pharmaceutical composition including at least one non-steroidal anti-inflammatory drug (NSAID) and at least one solubilizing agent to a body surface of a patient.

2. The method of inhibiting a biofilm of claim 1, wherein the at least one NSAID comprises at least one selected from the group consisting of acetylsalicylic acid, bucloxic acid, flufenamic acid, mefanamic acid, niflumic acid, tiaprofenic acid, tolfenamic acid, bendazac, carprofen, ketoprofen, diclofenac, diflunisal, etodolac, fenbufen, fenoprofen, fentiazac, flurbiprofen, isoxicam, naproxen, pirfrofen, piroxicam, sulindac, suprofen, tenoxicam, tolmetin, and zomepirac.

3. The method of inhibiting a biofilm of claim 1, wherein the at least one solubilizing agent comprises at least one ethoxylated oil.

4. The method of inhibiting a biofilm of claim 4, wherein the at least one ethoxylated oil includes a PEGylated oil including 9 to 24 ethoxylations.

5. The method of inhibiting a biofilm of claim 1, wherein the topical pharmaceutical composition comprises about 0.1% w/w to about 5% w/w of the at least one NSAID.

6. The method of inhibiting a biofilm of claim 3, wherein the topical pharmaceutical composition comprises about 1% w/w to about 20% w/w of the at least one ethoxylated oil.

7. The method of inhibiting a biofilm of claim 5, wherein the at least one solubilizing agent includes at least one ethoxylated oil and wherein the topical pharmaceutical composition comprises about 1% w/w to about 20% w/w of the at least one ethoxylated oil.

8. The method of inhibiting a biofilm of claim 5, wherein the topical pharmaceutical composition comprises about 2% w/w to about 5% w/w of the at least one NSAID.

9. The method of inhibiting a biofilm of claim 7, wherein the topical pharmaceutical composition comprises about 2% w/w to about 5% w/w of the at least one NSAID.

10. The method of inhibiting a biofilm of claim 6, wherein the topical pharmaceutical composition comprises about 5% w/w to about 10% w/w of the at least one ethoxylated oil.

11. The method of inhibiting a biofilm of claim 7, wherein the topical pharmaceutical composition comprises about 5% w/w to about 10% w/w of the at least one ethoxylated oil.

12. The method of inhibiting a biofilm of claim 9, wherein the topical pharmaceutical composition comprises about 5% w/w to about 10% w/w of the at least one ethoxylated oil.

13. The method of inhibiting a biofilm of claim 7, wherein the topical pharmaceutical composition is selected from the group consisting of an ointment, a cream, a gel, a lotion, a solution, and a paste.

14. The method of inhibiting a biofilm of claim 7, wherein about 500 mg/day to about 5000 mg/day of the topical pharmaceutical composition is applied to the body surface of the patient.

15. The method of inhibiting a biofilm of claim 14, wherein about 1000 mg/day to about 3000 mg/day of the topical pharmaceutical composition is applied to the body surface of the patient.

16. The method of inhibiting a biofilm of claim 12, wherein about 500 mg/day to about 5000 mg/day of the topical pharmaceutical composition is applied to the body surface of the patient.

17. The method of inhibiting a biofilm of claim 16, wherein about 1000 mg/day to about 3000 mg/day of the topical pharmaceutical composition is applied to the body surface of the patient.

18. A topical pharmaceutical composition comprising about 0.1% w/w to about 5% w/w of at least one non-steroidal anti-inflammatory drug (NSAID) including at least one selected from the group consisting of acetylsalicylic acid, bucloxic acid, flufenamic acid, mefanamic acid, niflumic acid, tiaprofenic acid, tolfenamic acid, bendazac, carprofen, ketoprofen, diclofenac, diflunisal, etodolac, fenbufen, fenoprofen, fentiazac, flurbiprofen, isoxicam, naproxen, pirfrofen, piroxicam, sulindac, suprofen, tenoxicam, tolmetin, and zomepirac and about 1% w/w to about 20% w/w of ethoxylated oils.

19. The topical pharmaceutical composition of claim 18 comprising about 2% w/w to about 5% w/w of the at least one non-steroidal anti-inflammatory drug (NSAID) and about 5% w/w to about 10% w/w of the ethoxylated oils.

20. The topical pharmaceutical composition of claim 19, wherein the ethoxylated oils are PEGylated oils including 9 to 24 ethoxylations.
